(19) United States
(12) Patent Application Publication
(10) Pub. No.: US 2024/0082356 A1
(43) Pub. Date: Mar. 14, 2024

(54) TREATMENT OF COPD BY STIMULATION OF STEM CELL MOBILIZATION

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); James VELTMEYER, Oceanside, CA (US)

(73) Assignee: Therapeutic Solutions International, Inc., Oceanside, CA (US)

(21) Appl. No.: 18/465,972

(22) Filed: Sep. 12, 2023

Related U.S. Application Data
(60) Provisional application No. 63/405,793, filed on Sep. 12, 2022.

(51) Int. Cl. A61K 38/19; A61K 31/09; A61K 35/28; A61P 11/00

(52) U.S. Cl. CPC A61K 38/193; A61K 31/09; A61K 35/28; A61P 11/00

(57) ABSTRACT

Disclosed are means of inducing pulmonary regeneration and/or protection from oxidative stress by stimulation of endogenous stem cell mobilization together with one or more inhibitors of NF-kappa B and/or one or more inhibitors of oxidative stress. The invention discloses the unexpected finding that G-CSF administration enhances oxidative stress and pulmonary damage, however, coadministration with pterostilbene, results in synergistic suppression of COPD pathology.

### CROSS-REFERENCE TO RELATED APPLICATIONS

This application claims priority to U.S. Provisional Application No. 63/405,793, titled “Treatment of COPD by Stimulation of Stem Cell Mobilization”, filed Sep. 12, 2022, which is incorporated by reference herein in its entirety.

### FIELD OF THE INVENTION

The teachings herein relate to the methods and compositions for treating chronic obstructive pulmonary disease utilizing stem cells.

### BACKGROUND

Chronic Obstructive Pulmonary Disease (COPD) is one of the major causes of morbidity and mortality. In the current state of medicine, COPD treatment consists primarily of addressing symptomology and not repairing the root cause of the disease.

In the lung, chronic obstructive pulmonary disease (COPD) that represents the third leading cause of death worldwide, is a pathology characterized by chronic inflammation, and has been recognized as an important risk factor for lung cancer. COPD is a heterogeneous pathology, comprised of two major components, obstructive bronchitis (small airways disease) and emphysema (parenchyma destruction). COPD is linked to an abnormal inflammatory immune response of the lung to noxious particle or gazes, and shares its main risk factor, tobacco, with lung cancer. The characteristics of COPD.sup.+ patients, when compared with smokers without COPD, is that they develop more pronounced destructive inflammation of the lung, starting with a strong release of TNF-.alpha. and CXCL-8 by epithelial cells and alveolar macrophages, leading to the recruitment of inflammatory monocytes and neutrophils. This results in extracellular matrix destruction and cell death through the release of matrix metalloproteinases (MMPs) and reactive oxygen species, ultimately resulting in emphysema. This chronic inflammation of the lung, accompanied by increased oxidant and noxious stress can lead to DNA adduct formation, and has logically been suggested to promote the earliest stages of carcinogenesis in COPD.sup.+ patients. Accordingly, the risk of lung cancer development in smokers with COPD is higher than the one in smokers without COPD. Genes involved in cell proliferation and survival, such as NF-.kappa.B and STAT3, that are up regulated in COPD (38) and activated by pro-inflammatory cytokines, including IL-6 and TNF-.alpha., play probably a major role in this process.

Previously attempts have been made to utilize regenerative cells in the lung, such as stem cells. Unfortunately, many of the stem cell approaches utilized do not see to yield significant benefit in Phase III trials. The current approach seeks to utilize endogenous stem cells through the process of mobilization.

### SUMMARY

Preferred embodiments are directed to methods of reducing lung pathology comprising administration of: a) a stem cell mobilizer; b) administration of an antioxidant; and c) optionally administering a stem cell population or several stem cell populations.

Preferred methods include embodiments wherein said lung pathology is chronic obstructive pulmonary disease (COPD).

Preferred methods include embodiments wherein said lung pathology is asthma.

Preferred methods include embodiments wherein said lung pathology is acute respiratory distress syndrome.

Preferred methods include embodiments wherein said lung pathology is idiopathic pulmonary fibrosis.

Preferred methods include embodiments wherein said lung pathology is lung cancer.

Preferred methods include embodiments wherein said lung pathology is bacterial infection.

Preferred methods include embodiments wherein said lung pathology is viral infection.

Preferred methods include embodiments wherein said lung pathology is fungal infection.

Preferred methods include embodiments wherein said stem cell mobilizer is G-CSF.

Preferred methods include embodiments wherein said stem cell mobilizer is GM-CSF.

Preferred methods include embodiments wherein said stem cell mobilizer is M-CSF.

Preferred methods include embodiments wherein said stem cell mobilizer is stem cell factor.

Preferred methods include embodiments wherein said stem cell mobilizer is flt-3 ligand.

Preferred methods include embodiments wherein said stem cell mobilizer is a CXCR4 inhibitor.

Preferred methods include embodiments wherein said stem cell mobilizer is a CXCL12 inhibitor.

Preferred methods include embodiments wherein said NF-kappa B inhibitor is pterostilbene.

Preferred methods include embodiments wherein said pterostilbene is administered as a nanoparticle.

Preferred methods include embodiments wherein said pterostilbene is administered as a liposome.

Preferred methods include embodiments wherein said pterostilbene is administered as an immunoliposome.

Preferred methods include embodiments wherein said pterostilbene is administered as an intravenous formulation.

Preferred methods include embodiments wherein said pterostilbene is administered as an intravenous formulation.

Preferred methods include embodiments wherein said cell population is autologous.

Preferred methods include embodiments wherein said cell population is allogeneic.

Preferred methods include embodiments wherein said cell population is xenogenic.

Preferred methods include embodiments wherein said cell population is bone marrow mononuclear cells.

Preferred methods include embodiments wherein said cell population is mesenchymal stem cells.

Preferred methods include embodiments wherein said cell population is amniotic stem cells.

Preferred methods include embodiments wherein said cell population is embryonic stem cells.

Preferred methods include embodiments wherein said cell population is inducible pluripotent stem cells.

Preferred methods include embodiments wherein said cell population is a hematopoietic stem cell population.

Preferred methods include embodiments wherein said mesenchymal stem cells express CD90.

Preferred methods include embodiments wherein said mesenchymal stem cells express CD105.

Preferred methods include embodiments wherein said mesenchymal stem cells express c-met.

Preferred methods include embodiments wherein said mesenchymal stem cells express CD133.

Preferred methods include embodiments wherein said mesenchymal stem cells express c-kit.

Preferred methods include embodiments wherein said mesenchymal stem cells express IL-1 receptor.

Preferred methods include embodiments wherein said mesenchymal stem cells express IL-1 receptor antagonist when treated with interferon gamma.

Preferred methods include embodiments wherein said interferon gamma is administered at a concentration of 5 ng/ml for a period of 1 hour to 24 hours.

Preferred methods include embodiments wherein said agents are administered together with a growth factor.

Preferred methods include embodiments wherein said growth factor is hepatocyte growth factor.

Preferred methods include embodiments wherein said growth factor is epidermal growth factor.

Preferred methods include embodiments wherein said growth factor is insulin growth factor.

Preferred methods include embodiments wherein said growth factor is keratinocyte growth factor.

### BRIEF DESCRIPTION OF THE DRAWINGS

FIG. 1 is a bar graph showing the reduction of neutrophil infiltration in COPD Model by G-CSF and Pterostilbene.

### DETAILED DESCRIPTION OF THE INVENTION

The invention teaches the use of endogenous stem cell mobilization together with one or more antioxidants and one or more inhibitors of NF-kappa B to reduce and/or revert pathology associated with COPD.

As used herein, the use of “or” means “and/or” unless stated otherwise. In the context of a multiple dependent claim, the use of “or” refers back to more than one preceding independent or dependent claim in the alternative only.

As used herein, the term “one or more” is readily understood by one of skill in the art, particularly when read in context of its usage.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “soluble P-selectin” refers to naturally occurring soluble form of P-selectin and recombinant form thereof, or polymorphic or allelic variant or other isoforms thereof. The term also comprises modified or unmodified soluble P-selectin, such as glycosylated or non-glycosylated forms.

As used herein, the terms “mobilize” and “mobilization” refer to processes by which a population of hematopoietic stem or progenitor cells is released from a stem cell niche.

As used herein, the term “hematopoietic stem cell” or “HSC” refers to a stem cell that is capable of differentiating into both myeloid lineages (i.e. monocytes, macrophages, neutrophils, basophils, eosinophils, erythrocytes, megakaryocytes/platelets and some dendritic cells) and lymphoid lineages (i.e. T-cells, B-cells, NK-cells, and some dendritic cells).

As used herein, the term “subject” refers to any animal, including mammals, birds, reptiles and amphibians and in preferred embodiments to mammals, including humans, companion animals, food production animals and wild animals.

The invention relates to the utilization of mobilizing agents together with antioxidants to inhibitor and/or reverse COPD pathology. In one embodiment mobilization of stem cells is achieved by the use of an adrenergic receptor agonist in combination with an agent that mobilizes hematopoietic stem cells or progenitor cells results in enhanced mobilization of the hematopoietic stem cells or progenitor cells from the bone marrow to the peripheral blood.

In one embodiment, the invention provides the use of agents that mobilize the hematopoietic stem cells or progenitors may act to decrease the expression or function of a chemokine (the function being the binding of the chemokine to its receptor and further signaling), particularly CXCL12. In another embodiment, the agents that mobilize the hematopoietic stem cells or progenitors may act to block or antagonize the chemokine receptor, CXCR4. In yet another embodiment, the agents that mobilize the hematopoietic stem cells or progenitor cells may act via an as yet unidentified mechanism. As such, the combination of these agents with an adrenergic receptor agonist may be used to treat a subject having cancer and who has undergone or is planning to undergo chemotherapy or irradiation therapy for a cancerous condition, whereby the subject will have reduced bone marrow cellularity due to the treatment regimen. As such, the combination of the adrenergic receptor agonists of the invention, when combined with, for example, a mobilizing therapy such as a colony stimulating factor like G-CSF, would be of significant value if it would allow for the use of lower levels of the colony stimulating factor, thus resulting in significant cost reduction to the patient, as well as perhaps, shortened hospital stays. Additionally, the methods of the present invention would allow for mobilization of the hematopoietic stem cells from the bone marrow to the circulation, thus allowing for collection of these cells from the patient prior to the onset of, for example, chemotherapy, to be administered back to the patient for autologous transplant.

The invention further teaches the use of other agents that act to increase white blood cells and/or hematopoietic stem cells or progenitor cells in both human and animal subjects. These include granulocyte-macrophage colony stimulating factor (GM-CSF), Interleukin-1 (IL-1), Interleukin-3 (IL-3), Interleukin-8 (IL-8), PIXY-321 (GM-CSF/IL-3 fusion protein), macrophage inflammatory protein (MIP), stem cell factor, thrombopoietin and growth related oncogene, as single agents or in combination (Dale, D., et al., Am. J. of Hematol. (1998) 57:7-15; Rosenfeld, C., et al., Bone Marrow Transplantation (1997) 17:179-183; Pruijt, J., et al., Cur. Op. in Hematol. (1999) 6:152-158; Broxmeyer, H., et al., Exp. Hematol. (1995) 23:335-340; Broxmeyer, et al., Blood Cells, Molecules and Diseases (1998) 24:14-30; Glaspy, J., et al., Cancer Chemother. Pharmacol. (1996) 38 (suppl): S53-S57; Vadhan-Raj, S., et al., Ann. Intern. Med. (1997) 126:673-81; King, A., et al., Blood (2001) 97:1534-1542; Glaspy, J., et al., Blood (1997) 90:2939-2951). However, while these agents are effective, there are known disadvantages to their use. For example, since many of these agents/growth factors are proteins, the effort put into the cloning, purification/isolation, in addition to the cost to the patient, sets the stage for searching for small molecule mimics that would be easier to manufacture and less costly for the patient in need of such therapy.

The combination of compounds proposed by the present invention may also enhance the success of bone marrow transplantation, and may also combat infections in the patient undergoing such therapies. The combination of compounds proposed are used to mobilize and harvest hematopoietic stem cells or progenitor cells via apheresis and the harvested cells are used in treatments requiring stem cell transplantations for treatment of COPD. Furthermore, the combination of one or more adrenergic receptor agonists with one or more mobilizing agents can be used both in vivo to promote mobilization of hematopoietic stem cells or progenitor cells from the bone marrow to the peripheral blood or can be used for ex vivo studies, whereby a patient's own stem cells are removed and expanded in culture for autologous transplants. Also contemplated by the present invention are in vitro screens, whereby candidate or test compounds can be measured for their effects on mobilization before being administered in vivo.

In some embodiments, adjuvants are added to increase mobilization of stem cells, said adjuvants may be selected from a list comprising of TNFR1, IL-1, IL-1R, IL-4, IL-4R, IL-5, IL-6, IL-6R, IL-8, IL-8R, IL-9, IL-9R, IL-10, IL-12 IL-12R, IL-13, IL-13R.alpha.1, IL-13R.alpha.2, IL-15, IL-15R, IL-16, IL-17R, IL-17, IL-18, IL-18R, IL-23 IL-23R, IL-25, CD2, CD4, CD11a, CD23, CD25, CD27, CD28, CD30, CD40, CD40 L, CD56, CD138, ALK5, EGFR, FcER1, TGFb, CCL2, CCL18, CEA, CRS, CTGF, CXCL12 (SDF-1), chymase, FGF, Furin, Endothelin-1, Eotaxins (e.g., Eotaxin, Eotaxin-2, Eotaxin-3), GM-CSF, ICAM-1, ICOS, IgE, IFNa, 1-309, integrins, L-selectin, MIF, MIP4, MDC, MCP-1, MMPs, neutrophil elastase, osteopontin, OX-40, PARC, PD-1, RANTES, SCF, SDF-1, siglec8, TARC, TGFb, Thrombin, Tim-1, TNF, TNFR1, TRANCE, Tryptase, VEGF, VLA-4, VCAM, .alpha.4.beta.7, CCR2, CCR3, CCR4, CCR5, CCR7, CCR8, alphavbeta6, alphavbeta 8, cMET, and CD8.

The use of stem cells for treatment of COPD has previously been described. We provide the following examples to guide one of skill in the art for administration of exogenous stem cells. The invention, however, provides the use of stem cell mobilization together with antioxidants and/or NF-kappa B inhibitors.

Numerous types of stem cells may be utilized for the practice of the invention. In one embodiment, Bone marrow may be collected and placed within a “washing vessel”. Before the collection procedure a “washing tube” is prepared in the class 100 Biological Safety Cabinet in a Class 10,000 GMP Clean Room. To prepare the washing tube, 0.2 mL amphotericin B (Sigma-Aldrich, St Louis, Mo.), 0.2 mL penicillin/streptomycin (Sigma 50 ug/nl) and 0.1 mL EDTA-Na2 (Sigma) is added to a 50 mL conical tube (Nunc) containing 40 mL of GMP-grade phosphate buffered saline (PBS). Specifically, the washing tube containing the collected bone marrow is topped up to 50 mL with PBS in a class 100 Biological Safety Cabinet and cells are washed by centrifugation at 500 g for 10 minutes at room temperature, which yields a cell pellet at the bottom of the conical tube. Under sterile conditions supernatant is decanted and the cell pellet is gently dissociated by tapping until the pellet appeared liquid. The pellet is re-suspended in 25 mL of PBS and gently mixed so as to produce a uniform mixture of cells in PBS. In order to purify mononuclear cells, 15 mL of Ficoll-Paque (Fisher Scientific, Portsmouth N.H.) density gradient is added underneath the cell-PBS mixture using a 15 mL pipette. The mixture is subsequently centrifuged for 20 minutes at 900 g. Thereafter, the buffy coat is collected and placed into another 50 mL conical tube together with 40 mL of PBS. Cells are then centrifuged at 400 g for 10 minutes, after which the supernatant is decanted and the cell pellet re-suspended in 40 mL of PBS and centrifuged again for 10 minutes at 400 g. The cell pellet is subsequently re-suspended in 5 mL complete DMEM-low glucose media (GibcoBRL, Grand Island, N.Y.) supplemented with 20% Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot is used for all experiments. Additionally, the media is supplemented with 1% penicillin/streptomycin, 1% amphotericin B, and 1% glutamine. The re-suspended cells are mononuclear cells substantially free of erythrocytes and polymorphonuclear leukocytes as assessed by visual morphology microscopically. Viability of the cells is assessed with trypan blue. Only samples with >90% viability were selected for cryopreservation in sealed vials. In some embodiments of the invention cells are expanded outside of the body. This may be to increase the number of cells and/or activity of the cells. Cell expansion for cells can be performed in a clean room facilities purpose built for cell therapy manufacture and meeting GMP clean room classification. In a sterile class II biologic safety cabinet located in a class 10,000 clean production suite, cells are thawed under controlled conditions and washed in a 15 mL conical tube with 10 ML of complete DMEM-low glucose media (cDMEM) (GibcoBRL, Grand Island, N.Y.) supplemented with 20% Fetal Bovine Serum (Atlas) from dairy cattle confirmed to have no BSE % Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used was sequestered and one lot was used for all experiments.

Cells were subsequently placed in a T-225 flask containing 45 mL of cDMEM and cultured for 24 hours at 37.degree. C. at 5% CO2 in a fully humidified atmosphere. This allowed the MSC to adhere. Non-adherent cells were washed off using cDMEM by gentle rinsing of the flask. Adherent cells are subsequently detached by washing the cells with PBS and addition of 0.05% trypsin containing EDTA (Gibco, Grand Island, N.Y., USA) for 2 minutes at 37.degree. C. at 5% CO2 in a fully humidified atmosphere. Cells are centrifuged, washed and plated in T-225 flask in 45 mL of cDMEM. In one embodiment this procedure results in generation of approximately 6 million cells per initiating T-225 flask. The cells of the first flask were then split into 4 flasks. Cells are grown for approximately 4 days after which approximately 6 million cells per flask were present (24 million cells total). All processes in the generation, expansion, and product production were performed under conditions and testing that was compliant with current Good Manufacturing Processes and appropriate controls, as well as Guidances issued by the FDA in 1998 Guidance for Industry: Guidance for Human Somatic Cell Therapy and Gene Therapy; the 2008 Guidance for FDA Reviewers and Sponsors Content and Review of Chemistry, Manufacturing, and Control (CMC) Information for Human Somatic Cell Therapy Investigational New Drug Applications (INDs); and the 1993 FDA points-to-consider document for master cell banks were all followed for the generation of the cell products described. In some embodiments, donor cells were collected in sterile conditions, shipped to a contract manufacturing facility, assessed for lack of contamination and expanded. The expanded cells were stored in cryovials of approximately 6 million cells/vial, with approximately 100 vials per donor. At each step of the expansion quality control procedures were in place to ensure lack of contamination or abnormal cell growth. In another aspect, cells are grown in media and the cells, along with the media, are recovered after about 5-10 days. The cells are prepared in this “conditioned” media for transfusion at concentrations of less than about 100,000 cells per mL Physiological electrolyte additives may be added. The cell solution is administered intravenously. In another embodiment, cells are grown in media for about 5-10 days. This media is then transfused intravenously without cells or given locally to the site of the injury. Further methods involve isolation and/or concentration of stem cell produced factors and/or further refinements of these chemicals and/or compounds.

### Example 1: Reduction of Neutrophil Infiltration in COPD Model by G-CSF and Pterostilbene

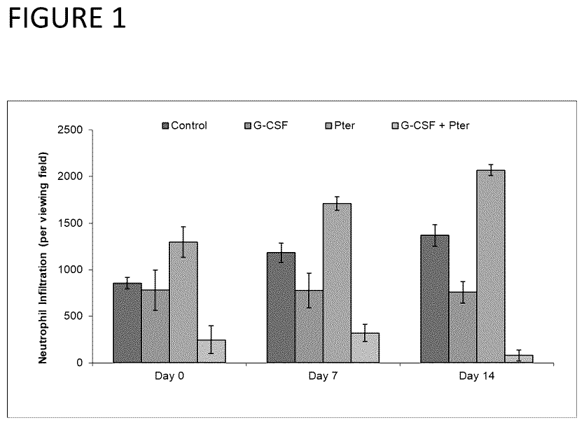

Female BALB/c mice, 10 per group, where injected with elastase (5 micrograms per mouse) to induce COPD like inflammation. Mice were given G-CSF (10 micrograms per mouse) every second day for one week and pterostilbene (40 mg/kg) intraperitoneally. Mice were sacrificed on days 0, 7, and 14 and assessed for neutrophil infiltration. Results are shown in FIG. 1.

1. A method of reducing lung pathology in a mammal comprising administration of: a) a stem cell mobilizer; b) administration of an antioxidant; and c) administering a stem cell population or several stem cell populations.

2. The method of claim 1, wherein said lung pathology is chronic obstructive pulmonary disease (COPD).

3. The method of claim 1, wherein said lung pathology is asthma.

4. The method of claim 1, wherein said lung pathology is acute respiratory distress syndrome.

5. The method of claim 1, wherein said lung pathology is idiopathic pulmonary fibrosis.

6. The method of claim 1, wherein said lung pathology is lung cancer.

7. The method of claim 1, wherein said lung pathology is bacterial infection.

8. The method of claim 1, wherein said lung pathology is viral infection.

9. The method of claim 1, wherein said lung pathology is fungal infection.

10. The method of claim 1, wherein said stem cell mobilizer is G-CSF.

11. The method of claim 1, wherein said stem cell mobilizer is GM-CSF.

12. The method of claim 1, wherein said stem cell mobilizer is M-CSF.

13. The method of claim 1, wherein said stem cell mobilizer is stem cell factor.

14. The method of claim 1, wherein said stem cell mobilizer is flt-3 ligand.

15. The method of claim 1, wherein said stem cell mobilizer is a CXCR4 inhibitor.

16. The method of claim 1, wherein said stem cell mobilizer is a CXCL12 inhibitor.

17. The method of claim 1, wherein said NF-kappa B inhibitor is pterostilbene.

18. The method of claim 17, wherein said pterostilbene is administered as a nanoparticle.

19. The method of claim 17, wherein said pterostilbene is administered as a liposome.

20. The method of claim 17, wherein said pterostilbene is administered as an immunoliposome.
